(19) United States
(12) Patent Application Publication
(10) Pub. No.: US 2026/0155055 A1
(43) Pub. Date: Jun. 4, 2026

(54) Computerized CBT Patient System

(71) Applicant: GAIA AG, Hamburg (DE)

(72) Inventors: Tristan Zindler, Hamburg (DE); Olaf Nackenhorst, Dortmund (DE); Bernhard Wellhöfer, Hamburg (DE); Mario Weiss, Hamburg (DE)

(21) Appl. No.: 18/966,803

(22) Filed: Dec. 3, 2024

(51) Int. Cl. G09B 5/02; G16H 10/60; G16H 80/00

(52) U.S. Cl. CPC G09B 5/02; G16H 10/60; G16H 80/00

(57) ABSTRACT

A cognitive behavioral therapy training companion is provided that includes: a character trait database having a plurality of human character traits; a message database having a plurality of patient memories; a computer with access to the character trait database and the message database, said computer having a communication link between said computer and a therapist; software executing on the computer for retrieving a patient memory and a set of character traits for a test patient and formulating an inner voice of the test patient based on the patient memory and the character traits; software executing on the computer for combining a new message with the inner voice to compose a reply of the test patient; software executing on the computer to update the patient memory based on the new message and the reply; and transmitting the reply to the therapist using the communication link.

### TECHNICAL FIELD

The present disclosure relates to computers that are configured to provide interactive dialog experiences (“patients”), and more particularly, to improvements in the algorithms that patients use to provide such experiences.

### BACKGROUND

Presently, generative artificial intelligence systems (“GenAI”) are prevalent. Such systems use statistical guessing to produce a most likely correct reply to a prompt. They lack rigor in the algorithms that generate their replys, and they are prone to mistaken guesses called “hallucinations”. For specialized tasks that an LLM is not specifically trained on, there is not a high likelihood that a particular reply will be “correct” in a useful sense of that term.

An example of a specialized task is the training of Cognitive Behavioral Therapy (“CBT”) therapists. CBT sometimes may be referred to as “talk therapy.” In the CBT treatment modality, a patient or client converses with a trained professional to enhance the patient's functioning with any of a range of psychological disorders including depression, PTSD, etc.

Treatment sessions are traditionally held in person in a comfortable setting between therapist and patient in order to promote communication and engagement. The treatment seeks to help patients become more self-aware and recognize factors that influence their emotional well-being, and also to encourage supportive behaviors and activities so that patients can reach and maintain their own emotional balance.

It is generally known that a series of regular CBT sessions is necessary to reinforce treatment initiatives until patients themselves become aware of improvements in their emotional state.

Ubiquitous Internet connectivity and the rise of mobile computing devices have made it possible for CBT to be consumed by patients without visiting a therapists'office. The patient's own environment and schedule can be more easily accommodated to not only leverage their personal comfort, but also to expand delivery of CBT services.

Training of CBT practitioners generally involves a trainee shadowing an experienced practitioner during treatment of an actual patient. But with the expanding demand for talk therapy, not enough experienced practitioners are available for trainees to get their hours. This presents a chicken/egg problem where the pool of practitioners cannot be expanded fast enough because the pool of practitioners is not big enough. It would be desirable to have a way for a CBT trainee to practice their craft without needing an experienced practitioner to supervise the trainee working with a real patient.

Computerized therapy systems are known, including systems for providing CBT, but these systems are only “intelligent” in the sense that they have the ability to answer a limited number of questions or provide a limited amount of information. Additionally, such systems have been only text-based. They either cannot accept inputs other than text, or they only provide replies in text, or both. So, the efficacy of existing computerized CBT systems is limited at least because the full range of a therapists'observations and experience cannot be used for treatment.

Further, current generation computerized therapy systems, including systems which may be operated as “patients,” have trouble with logic and reasoning because they are fundamentally statistical guessing machines that produce the most “likely” reply to a prompt. When handling signals of intense emotional valence, without some kind of framework for understanding the signals, the potential for over-simplification by an unsupervised computerized therapy system could be counter-productive or even dangerous.

### SUMMARY

According to aspects of the present disclosure, a cognitive behavioral therapy training companion is provided that includes: a character trait database having a plurality of human character traits; a message database having a plurality of patient memories; a computer with access to the character trait database and the message database, said computer having a communication link between said computer and a therapist; software executing on the computer for retrieving a patient memory and a set of character traits for a test patient and formulating an inner voice of the test patient based on the patient memory and the character traits; software executing on the computer for combining a new message with the inner voice to compose a reply of the test patient; software executing on the computer to update the patient memory based on the new message and the reply; and transmitting the reply to the therapist using the communication link.

Thus, aspects of the present disclosure can provide a patient that is available 24/7 for use by CBT trainees. The system can be realized through a mobile text interface, for example, by texting a given number. Given the capabilities of speech-to-text and text-to-speech, as well as the ability for speaking video generation from 2-D still images and text, voice and video interfaces also are contemplated.

Such a patient can provide trainees timely and consistent support, regardless of time or location. By using advanced agent-based systems to deliver personalized replys, the patient can focus on the individualized needs of trainees, enhancing the accessibility and effectiveness of support.

Embodiments of a patient according to the present disclosure are not limited to a specific mode of communication. Such a patient can support various communication platforms, such as a proprietary web app, WhatsApp, SMS (Simple Message Service), RCS (Rich Communication Services), iMessages, Signal, Face Time or other text, voice, and/or video modalities. Thus, a patient according to aspects of the present disclosure may allow trainees to choose their preferred communication method. Speech-to-text, text-to-speech, and text-to-video technologies enable consistent and seamless interaction across different platforms, and enhance accessibility by catering to diverse user preferences and needs. The disclosed patient delivers a cohesive user experience regardless of the communication channel used.

A multi-agent approach is a key aspect of the present disclosure. In the patient interaction, each reply is computed not in a single step but through a complex interplay of multiple agents. These agents distribute intermediate “cognitive” steps across multiple specialized requests to generate a supportive reply. Each agent is specialized in handling specific aspects of the reply-generation task, contributing to a more accurate and efficient overall reply. The system can adapt to different support scenarios by reconfiguring the agents and their interactions. By distributing tasks among multiple agents, the system enhances resilience and fault tolerance, reducing the impact of any single point of failure. Specialized agents improve the likelihood that each aspect of the support algorithm is addressed with the highest level of expertise, improving the overall accuracy and effectiveness.

Key agents include a memory, a character traits database, an inner voice, and a composer.

The memory is configured to generate a narrative from a series of messages and replies. Thus, the memory forms a summary of the case or conversation between the patient and the trainee. Overall, the memory provides a long-term memory representation of the patient's interaction with the trainee. The memory of the digital patient is generated by an LLM agent by summarizing the conversation with the trainee/therapist from the patient's perspective. The summarized or compressed information enables maintenance of continuity in the conversation by keeping track of the trainee's history, attributes, progress. The memory's representation of the interaction also enables provision of insight into the interaction. The memory operates in parallel to the other agents, so that its algorithm does not drive latency in the conversation.

The inner voice is a representation of the patient's inner monologue, which expresses the struggle, thoughts and feelings the patient has during therapy. Thus, the inner voice utilizes information from the memory and from the character traits database. The inner voice maintains consistency in the conversation.

The composer is configured to represent the cognitive process of a professional's client, e.g., a behavioral treatment therapy client. As such, the composer generates the test patient's reply to a new message from the trainee by combining the new message with the inner voice representing the current state of mind of the patient. However, the memory and inner voice of the patient are updated asynchronously and thus do not delay the generation of the reply. Once the composer formulates a reply, the reply is stored in the patient's memory and is transmitted to the trainee via the communication link. The composer, by operating in parallel to the other agents to plan the course of the conversation, enhances reply speed from the trainee's perspective.

Other features and aspects of the present teachings will become apparent from the following detailed description, taken in conjunction with the accompanying drawings, which illustrate by way of example the features in accordance with embodiments of the present teachings. The summary is not intended to limit the scope of the present teachings.

### BRIEF DESCRIPTION OF THE DRAWINGS

The present teachings are described more fully hereafter with reference to the accompanying drawings, which depict example embodiments. The following description illustrates the present teachings by way of example, not by way of limitation of the principles of the present

### Teachings.

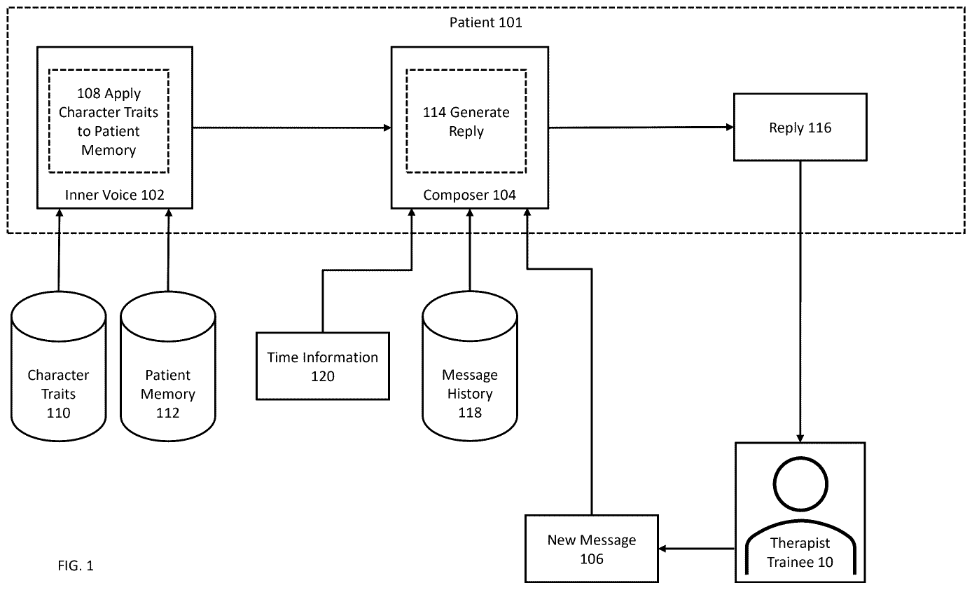

FIG. 1 depicts an overall interaction 100 of a patient 101 with a trainee 10, consistent with selected aspects of the disclosure.

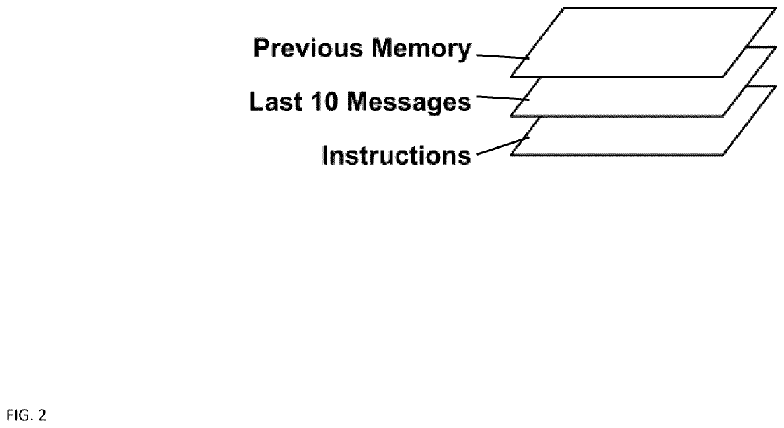

FIG. 2 depicts layers of the patient 101 patient memory 112.

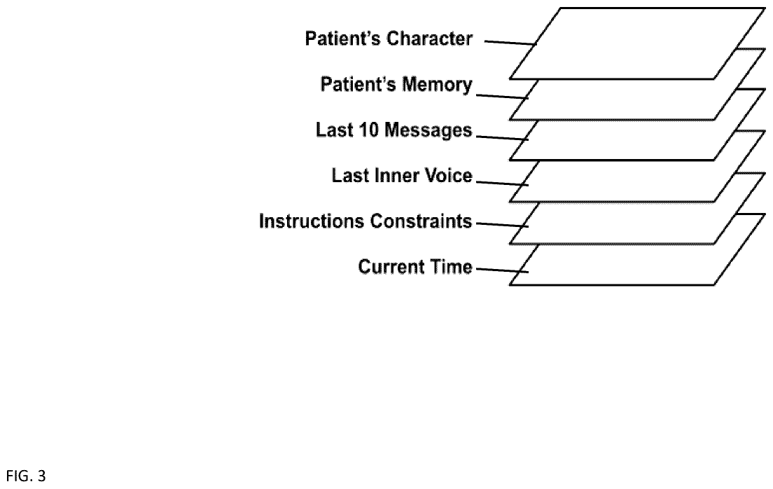

FIG. 3 depicts inputs to a prompt 300 for a composer 104 of the patient 101.

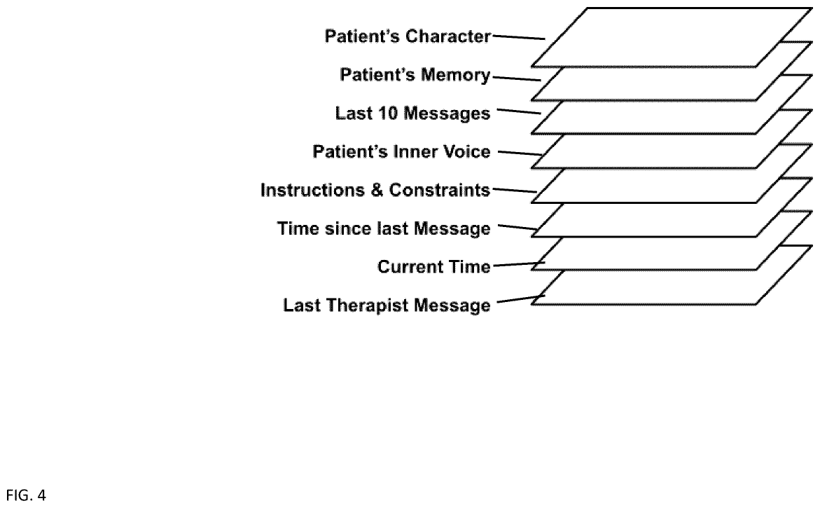

FIG. 4 depicts inputs to a prompt 400 for an inner voice 102 of the patient 101.

It should be understood that throughout the drawings corresponding reference numerals indicate like or corresponding parts and features.

### DETAILED DESCRIPTION

For purposes of explanation and not limitation, specific details are set forth such as particular structures, architectures, interfaces, techniques, etc. in order to provide a thorough understanding. In other instances, detailed descriptions of well-known devices and/or methods are omitted so as not to obscure the description with unnecessary detail.

FIG. 1 depicts an overall interaction 100 of a CBT training companion (“patient”) 101 with a therapist trainee 10, consistent with selected aspects of the disclosure. The patient 101 includes an inner voice 102 and a composer 104. The patient receives a new message 106 from a therapist trainee and produces a reply 116.

First the patient 101 formulates the inner voice 102 by applying 108 character traits 110 to a patient memory 112. For example, the inner voice 102 uses the character traits 110 as a context portion of a complex LLM prompt and uses the patient memory 112 as a situation portion of the complex LLM prompt where the task is to produce the inner voice 102: (“You are a person who [character traits=context] and you have had the following conversations: [=situation]. Tell me what you are thinking [=task].”). For example, the set of character traits 110 may be in the form of a complex (many token, e.g., thousands of tokens) prompt. Alternatively, the character traits 110 may be encoded in the weights of a neural network in the patient 101.

Next, the composer 104 generates 114 the reply 116, based at least on the inner voice 102, the new message 106, a history of messages 118, and time information 120. The composer 104 applies the inner voice 102 to the new message 106 in order to generate the reply 116. For example, the composer 104 may use the inner voice 102 as a context portion of a complex LLM prompt and may use the new message 106 as a situation portion of the complex LLM prompt where the task is to produce the reply 116: (“You are thinking [inner voice 102=context] and the therapist says [new message 106=situation]. What do you say next? [=task]”).

At each iteration of new message 106 and reply 116, the patient 101 stores these communications in the patient memory 112. FIG. 2 depicts how the patient 101 stores communications in the memory 112. The layers of the “memory” 112 include previous memory (a compressed or encoded version of the patient memory 112 plus character traits 110), the last ten or however many messages (ten messages being an example buffer window for convenience of understanding), and instructions (e.g., the current character traits 110). The memory 112 is used as part of a prompt to generate the inner voice 102. Purposes of the memory include compressing the therapy conversation, mitigating “whispers”, extracting details from the conversation, tracking trainee experience and patient progress, goal and task tracking, and identification of information gaps.

FIG. 3 depicts inputs to a prompt 300 for the inner voice 102 of the patient 101. Purposes of the inner voice 102 include creating a patient's inner monologue; emulating cognition of the patient; expressing struggle, thoughts, and feelings; maintaining character consistency across messages 116; maintaining continuity of the conversation; and keeping aware of time. The prompts include the character traits 110, the memory 112, the last ten messages, the last composer, instructions and constraints, and a current time. Each iteration of the composer updates the previous composer.

FIG. 4 depicts inputs to a prompt 400 for the composer 104 of the patient 101. The purpose of the composer is to produce a “realistic” reply 116 as a patient's answer to the CBT trainee's new message 106. Instructions and constraints for the composer include language and style; structure and content; contextual awareness; interaction dynamics; and behavioral realism. Accordingly, the composer 104 incorporates the character traits 110, the memory 118, the last ten messages, the inner voice 102, the aforementioned instructions and constraints, a time since the last message, a current time, and the new message 106.

A prototype of the patient operates on multiple instances of GPT-4 by OpenAI. Open-source models such as LLAMA 3 are equally suitable. The patient may be self-hosted. Using multiple instances of large language models (LLMs) that take separate customized prompts and/or are trained on custom data enables the patient 101 to produce high-quality replys. LLMs can provide powerful capabilities for processing and generating human-like text. Moving to open-source models may enhance scalability and provide greater control over the system. For example, using a self-hosted open-source model may allow for customization and fine-tuning to meet specific support needs. Additionally, self-hosting ensures higher security and better privacy for user data.

The present teachings have been described in language more or less specific as to structural, mechanical, and functional features. It is to be understood, however, that the present teachings are not limited to the specific features shown and described, since the apparatus, system, and/or method herein disclosed comprises preferred forms of putting the present teachings into effect.

Generally, all terms used in the claims are to be interpreted according to their ordinary meaning in the technical field, unless explicitly defined otherwise herein. All references to a/an/the element, apparatus, component, means, step, etc. are to be interpreted openly as referring to at least one instance of the element, apparatus, component, means, step, etc., unless explicitly stated otherwise. The use of “first”, “second,” etc. for different features/components of the present disclosure are only intended to distinguish the features/components from other similar features/components and not to impart any order or hierarchy to the features/components, unless explicitly stated otherwise. The phrase “at least one of,” when used with a list of items, means that different combinations of one or more of the listed items may be used, and only one item in the list may be needed. For example, “at least one of: A, B, and C” includes any of the following combinations: A; B; C; A and B; A and C; B and C; and A and B and C.

Other than in the operating examples, or where otherwise indicated, all numbers expressing quantities of ingredients or reaction conditions used herein are to be understood as modified in all instances by the term “about”.

While the present teachings have been described above in terms of specific embodiments, it is to be understood that they are not limited to those disclosed embodiments. Many modifications and other embodiments will come to mind to those skilled in the art to which this pertains, and which are intended to be and are covered by both this disclosure and the appended claims. For example, in some instances, one or more features disclosed in connection with one embodiment can be used alone or in combination with one or more features of one or more other embodiments. It is intended that the scope of the present teachings should be determined by proper interpretation and construction of any claims and their legal equivalents, as understood by those of skill in the art relying upon the disclosure in this specification and the attached drawings.

1. A cognitive behavioral therapy training companion system comprising:
a character trait database having a plurality of human character traits;
a message database having a plurality of patient memories;
a computer with access to the character trait database and the message database, said computer having a communication link between said computer and a therapist;
a first distinct software module executing on the computer for periodically retrieving a patient memory and a set of character traits for a test patient and formulating an inner voice of the test patient based on the patient memory and the character traits;
a second distinct software module executing on the computer for combining a new message with the inner voice to compose in real time a reply of the test patient and transmit the reply to the therapist using the communication link; and
a third distinct software module executing on the computer to update the patient memory based on the new message and the reply;
wherein the formulation of the inner voice, the composition and transmission of the reply, and the update of the patient memory are asynchronous from each other.

2. The system of claim 1, wherein the composer software incorporates the character traits, the emotional summary, and the last ten messages.

3. The system of claim 1, wherein the composer software incorporates instructions and constraints related to at least one of language and style; structure and content; contextual awareness; interaction dynamics; and behavioral realism.

4. The system of claim 1, wherein each message history is a series of messages and replies, further comprising a memory that is configured to generate a narrative from a message history.

5. The system of claim 4, wherein the memory compresses the narrative into a compact mathematical representation.

6. The system of claim 4, wherein the composer software uses the narrative to maintain continuity of communication.

7. The system of claim 4, wherein the inner voice software uses the narrative to provide insight into the interaction.

8. The system of claim 4, wherein the composer generates an assessment of the interaction and the therapist by analyzing the narrative in combination with character traits.

9. The system of claim 4, wherein the memory incorporates instructions for tracking trainee experience.

10. The system of claim 4, wherein the memory incorporates instructions for tracking patient progress.

11. The system of claim 4, wherein the memory incorporates instructions for tracking goals and/or tasks.

12. The system of claim 11, wherein the composer generates a message based on the memory tracking a goal or task as not completed.

13. The system of claim 4, wherein the memory incorporates instructions for identifying information gaps.

14. The system of claim 13, wherein the composer generates a message based on the memory identifying an information gap.

15. A non-transitory computer readable medium that is encoded with instructions, which, when executed by a computer, implement a system comprising:
a character trait database having a plurality of human character traits;
a message database having a plurality of patient memories;
a computer with access to the character trait database and the message database, said computer having a communication link between said computer and a therapist;
a first distinct software module executing on the computer for periodically retrieving a patient memory and a set of character traits for a test patient and formulating an inner voice of the test patient based on the patient memory and the character traits;
a second distinct software module executing on the computer for combining a new message with the inner voice to compose in real time a reply of the test patient and transmit the reply to the therapist using the communication link; and
a third distinct software module executing on the computer to update the patient memory based on the new message and the reply;
wherein the formulation of the inner voice, the composition and transmission of the reply, and the update of the patient memory are asynchronous from each other.
